(19) United States
(12) Patent Application Publication
(10) Pub. No.: US 2021/0078056 A1
(43) Pub. Date: Mar. 18, 2021

(54) METHOD FOR TRANSFORMING ARSENIC SULFIDE SLAG AND CURING AND STABILIZING RESULTING COMPOUND BY MEANS OF MICROENCAPSULATION

(71) Applicant: BEIJING GEOENVIRON ENGINEERING & TECHNOLOGY, INC, Beijing (CN)

(72) Inventors: Chengli HUO, Beijing (CN); Hui XIONG, Beijing (CN); Liqi LIU, Beijing (CN); Zejun LIU, Beijing (US); Shengli ZHEN, Beijing (CN); Zhen HE, Beijing (CN); Chao LIU, Beijing (CN)

(73) Assignee: BEIJING GEOENVIRON ENGINEERING & TECHNOLOGY, INC, Beijing (CN)

(21) Appl. No.: 16/971,312

(22) PCT Filed: Dec. 10, 2018
(86) PCT No.: PCT/CN2018/120017

§ 371 Date: Aug. 20, 2020

(30) Foreign Application Priority Data
Nov. 26, 2018 (CN) 201811417846.X

(51) Int. Cl. B09B 3/00; B01J 13/04; C12N 11/14; C09D 7/62; C01B 33/12; C01B 37/02

(52) U.S. Cl. CPC B09B 3/00; B01J 13/04; C12N 11/14; B82Y 30/00; C01B 33/126; C01B 37/02; C09D 7/62

(57) ABSTRACT

The present disclosure provides a method for transforming an arsenic sulfide slag and curing and stabilizing the resulting compound by means of microencapsulation, comprising the following steps: (1) preparing arsenic trioxide from the arsenic sulfide slag as a raw material; (2) preparing 4-hydroxy-3-nitrophenylarsonic acid from the arsenic trioxide as a raw material; (3) preparing an iron-manganese dinuclear cluster metal arsenate compound having a porous structure; (4) subjecting the iron-manganese dinuclear cluster metal arsenate compound having a porous structure to surface coating with silicon; (5) synthesizing an Fe(0)/Al-SBA-15 mesoporous composite stabilizer by a hydrothermal reaction; and (6) subjecting the silicon coated iron-manganese dinuclear cluster metal arsenate compound to curing and stabilizing treatment by means of microencapsulation. The present disclosure involves transforming the arsenic sulfide slag into 4-hydroxy-3-nitrophenylarsonic acid and finally into a metal arsenate compound having a porous structure, which has the characteristics of good stability and low toxicity in comparison to conventional arsenic compounds. Thus, the toxicity associated with arsenic compounds can be greatly reduced.

### FIELD OF THE DISCLOSURE

The present disclosure relates to a method for treating an arsenic sulfide slag, particularly, to a method for transforming an arsenic sulfide slag and curing and stabilizing the resulting compound by means of microencapsulation, which pertains to the technical field of hazardous waste treatment, suitable for treatment of arsenic-containing waste.

### BACKGROUND OF THE DISCLOSURE

In the process of chemical and metallurgical production, a large amount of high arsenic acid is usually produced. The arsenic in the waste liquid is usually removed by a sulfide precipitation method to obtain arsenic sulfide slag. At present, curing and stabilization is a technology commonly used at home and abroad to treat arsenic sulfide slag. Inorganic materials such as cement and lime are usually used for the curing. Due to the large amount of cement added, the product after the curing has a large compatibilization ratio, resulting in higher disposal costs.

Patent CN102151690A discloses a method for treating arsenic sulfide slag by adding an inorganic flocculant liquid to an arsenic sulfide slag, stirring the same uniformly, then adding a solid powder adsorbent, and finally adding asbestos wool to stir, so that the leaching toxicity of arsenic can meet the requirements of hazardous waste field. However, the solidified arsenic slag has a poor long-term stability.

An arsenic sulfide slag generally contains valuable metals, such as Cu, Bi, etc., but when recovering valuable metals, arsenic is generally recovered first due to high arsenic content of the arsenic sulfide slag. Patent CN103388076A discloses a method for recovering elemental arsenic from an arsenic sulfide slag. Through an oxidative desulfurization leaching-acidification reduction process, elemental arsenic with arsenic purity greater than 98% is obtained, and the recovery rate reaches 99%. However, the elemental arsenic is of low purity and it is subject of surface oxidization, which limits application of this method. Patent CN107012340A discloses a full wet process for extracting arsenic from an arsenic sulfide slag, wherein the arsenic sulfide slag is leached by oxygen pressure and through solid-liquid separation are a sulfur slag and a leachate containing pentavalent arsenic and sulfuric acid obtained. The arsenic sulfide slag is used as a reductant agent to reduce the pentavalent arsenic, and the solution of trivalent arsenic is obtained by solid-liquid separation. After cooling, crystallizing and drying, an arsenic white product is obtained, which, however, is highly toxic.

At present, none of the methods for treatment and disposal of arsenic sulfide slags has solved the long-term stability of arsenic slag solidification and the toxicity of arsenic after recovery. Therefore, it is of great practical significance to develop a method of transforming an arsenic sulfide slag to prepare low-toxicity, high-stability arsenic compounds, and at the same time bringing about efficient curing and stabilization effects.

### SUMMARY OF THE DISCLOSURE

Upon the problem existing in the treatment of an arsenic sulfide slag at present, the object of the present disclosure is to provide a method for transforming an arsenic sulfide slag and curing and stabilizing a resulting compound by means of microencapsulation.

In order to achieve the object, the present disclosure is achieved through the following technical solutions:

A method for transforming an arsenic sulfide slag and curing and stabilizing a resulting compound by means of microencapsulation, including the following steps:

First step: preparing arsenic trioxide from the arsenic sulfide slag.

First, the arsenic sulfide slag is added into a 50% concentration (mass fraction) sulfuric acid solution with a liquid-to-solid ratio (mass ratio) of 5:1, and stirred in a slurry tank for slurrying, with a stirring speed of 300-500rpm/min and a stirring time of 1-2 h. After the slurrying, a slurry is pumped into a high-pressure reaction vessel, and a 70% concentration (mass fraction) sulfuric acid solution is added to adjust a liquid-solid ratio (mass ratio) in the reaction vessel into 7:1, and a temperature in the reaction vessel is 150-160° C. Oxygen is introduced into the reaction vessel, with an oxygen partial pressure controlled to be 0.6-0.7 MPa, and the arsenic sulfide slag is oxdative pressure leached, with leaching reaction time of 3-4 h. After the leaching reaction is completed, filtration is performed to achieve solid-liquid separation, and filtrate is pumped into a closed reaction vessel, and then reduced with sulfur dioxide as introduced, and cooled and crystallized after the reduction, and arsenic trioxide is obtained through filtration and purification.

Second step: preparing 4-hydroxy-3-nitrophenylarsonic acid from the arsenic trioxide.

\hat{1} A little excess of aniline is added into a microwave heating reaction vessel and heated to 60-70° C. . Later, arsenic acid solution produced by reaction of the arsenic trioxide prepared in the first step with 0.5 mol/L excess hydrogen peroxide is uniformly added to the reaction vessel, and the heating is continued up to 165-180° C. for high-temperature synthesis of arsanilic acid.

\hat{2} Purification of the arsanilic acid: firstly, adding a 1 mol/L sodium hydroxide solution to the arsanilic acid generated at high temperature in the step CD for alkaline layering, with a liquid-to-solid ratio (mass ratio) of 2:1; removing waste aniline remaining after the high-temperature synthesis reaction in removed floating liquid, and then adding an appropriate amount of hydrochloric acid with a concentration of lmol/L to neutralize the solution to a pH value of 3.5-5.0; adding a certain amount of water to achieve a volume ratio 1:2 of water to hydrochloric acid, and meanwhile heating the solution to boiling at 100-105° C. for hydrolysis to remove a by-product caused by high-temperature synthesis of the arsanilic acid; after the hydrolysis is completed, moving the solution to a crystallization tank to be cooled and crystallized at 0-10° C.; after the arsanilic acid is fully crystallized, performing filtration and then crushing a filter cake and adding water again to produce a slurry, with a liquid-solid ratio 2:1 of water to arsanilic acid (mass ratio), and adding 0.1 mol/L sodium hydroxide solution to adjust a pH value to 6-7, and meanwhile heating the solution to 95° C., adding activated carbon for decolorization and impurity removal, and after completion of the impurity removal, cooling the solution to crystallize and freeze-dried at −10° C. to obtain arsanilic acid.

\hat{3} Synthesis of 4-hydroxy-3-nitrophenylarsonic acid: adding the arsanilic acid obtained above into a reaction vessel, first adding concentrated nitric acid (generally 68%-70% by mass), adjusting temperature, and then gradually adding sodium nitrite solution for diazotization at a certain temperature, wherein the diazotization temperature is 0-10° C., and wherein a molar ratio of arsenic acid, nitric acid and sodium nitrite is 5:6:0.7; hydrolyzing and nitrating the solution after the diazotization is completed; holding the reaction at a constant temperature for lh when the temperature rises to 55-75° C.; continuing to rise the temperature to 95-115° C. after nitrogen is surely released, and then stopping the temperature rising; and performing cooling and crystallization after completion of the hydrolysis and nitration, and precipitating a supernatant after the crystallization is completed, and obtaining 4-hydroxy-3-nitrophenylarsonic acid through suction filtration, freeze-drying, and pulverization.

Third step: preparing an iron-manganese dinuclear cluster metal arsenate compound having a porous structure.

10 mL of 0.1 mol/L hydrated iron perchlorate solution, 10 mL of 0.1 mol/L hydrated manganese perchlorate solution, and 10 mL of 0.1 mol/L complex solution are mixed, and then a 20 mL hot water solution of 0.2 mol/L 4-hydroxy-3-nitrophenylarsonic acid, with a temperature of 80° C., is added, and for example a hydrochloric acid solution having a concentration of 3 M is added and stirred to adjust a pH value to 3.5-5.5, and then 2 g of template is added to adjust temperature of the solution to 60° C., and stirred for 24 hours for sol-gel reaction, and then evaporated in an oven at 80° C., and final dried gel is calcined at a certain temperature at a heating rate of 2° C. /min for 4 hours, preferably at a temperature of 300-400° C., to obtain an iron-manganese dinuclear cluster metal arsenate compound having a porous structure.

Fourth step: subjecting the iron-manganese dinuclear cluster metal arsenate compound having a porous structure to surface coating with silicon.

The iron-manganese dinuclear cluster metal arsenate compound having a porous structure is uniformly dispersed into a 1:1 alcohol aqueous solution, with a liquid-to-solid ratio (mass ratio) of 10:1, and stirred at a speed of 800 r/min in a 40° C. water bath, and a surface coating agent is added drop by drop, wherein mass ratio of the surface coating agent to the metal arsenate compound is 1:50, and the stirring speed is increased to 1000 r/min and kept for 1 hour. After completion thereof, suction filtration, washing, drying at a temperature of 80° C. and grinding are performed to obtain an iron-manganese dinuclear cluster metal arsenate compound having a porous structure that is surface coated with silicon.

Fifth step: preparing an Fe(0)/Al-SBA-15 mesoporous composite stabilizer.

\hat{1} First, preparation of Al-SBA-15 mesoporous material: weighing a certain amount of template and surfactant to dissolve in 100 mL of deionized water, stirring the same in a 40° C. water bath to achieve uniform dissolution, and then weighing a certain mass of aluminum nitrate and adding the same to the above solution to dissolve evenly and remain stable, and adding ethyl orthosilicate to the above solution, keeping stirring for 48 hours in a 40° C. water bath, and then transferring a reaction product to a reaction vessel for hydrothermal reaction for 24 hours at a temperature of 105-115° C., and finally performing suction and filtration, washing, drying and calcining at a temperature of 650° C. at a heating rate of 2° C./min for 6 hours to obtain Al-SBA-15 mesoporous material, wherein the template, the surfactant, the aluminum nitrate and the ethyl orthosilicate have a mass ratio of 40:20:1:40.

\hat{2} Preparation of an Fe(0)/Al-SBA-15 mesoporous composite stabilizer: fully dissolving a certain amount of ferrous sulfate into 100 mL of ethanol aqueous solution, then adding Al-SBA-15 mesoporous material powder in the above solution to be ultrasonically dispersed for 5 minutes, continuing to stir for 20 hours after completion thereof, and then transferring the same to a 500 mL three-necked flask, and adding nitrogen gas for 15 minutes to remove dissolved oxygen; then adding polyethylene glycol 4000 to the above solution, stirring it for 30 minutes, and adjusting a pH value to 6 with 1M NaOH solution; and finally, under continuous stirring, adding dropwise (1 drop/sec) 0.1 mol/L reductant aqueous solution using a separatory funnel, and continuing the reaction for 40 minutes after the dropping, wherein a mass ratio of the ferrous sulfate, the Al-SBA-15 mesoporous material powder, the polyethylene glycol 4000, and the reductant agent is 4:4:1:2; after the reaction, when the mixture deposits to a bottom of a bottle, a precipitate is separated by centrifugation, and the precipitate obtained is washed alternately with deoxygenated deionized water and deoxygenated absolute ethanol for 3 times, dried and cooled in a vacuum drying oven at 70° C. to obtain the Fe(0)/ Al-SBA-15 mesoporous composite stabilizer.

Sixth step: curing and stabilizing by means of microencapsulation.

55-65 parts by mass of iron-manganese dinuclear cluster metal arsenate compound having a porous structure to surface coating with silicon, 10-15 parts by mass of Fe(0)/Al-SBA-15 mesoporous composite stabilizer, 8-12 parts by mass of immobilizing agent, and 3-5 parts by mass of immobilized enzyme are weight for curing and stabilization at 25° C., and a cured body is cured at 30° C. for 3 days.

Preferably, the complexing solution in the third step is an ammonium citrate solution.

Preferably, the template in the third step is F127.

Preferably, the surface coating agent in the fourth step is 3-aminopropyltriethoxysilane.

Preferably, in the fifth step, the template is P123, and the surfactant is polyethylene glycol 4000.

Preferably, the reductant in the fifth step is potassium borohydride.

Preferably, the immobilizing agent in the sixth step is rectorite powder, and further preferably, the rectorite powder has an average particle size of 5 μm.

Preferably, the immobilized enzyme in the sixth step is TerraZyme bio-immobilized enzyme.

The method for transforming an arsenic sulfide slag and curing and stabilizing the resulting compound by means of microencapsulation provided by the present disclosure has the following positive effects:

(1) The method for transforming an arsenic sulfide slag and curing and stabilizing the resulting compound by means of microencapsulation provided by the present disclosure uses an arsenic sulfide slag as a raw material to prepare 4-hydroxy-3-nitrophenylarsonic acid through transformation, and finally prepare porous structure. Compared with traditional arsenic compounds, metal arsenate compounds have the characteristics of better stability and lower toxicity, which greatly reduces the toxicity of arsenic compounds.

(2) The method for transforming an arsenic sulfide slag and curing and stabilizing the resulting compound by means of microencapsulation provided by the present disclosure, synthesizing an Fe(0)/Al-SBA-15 mesoporous composite material as a stabilizer, screens rectorite powder and liquidous TerraZyme (Terran enzyme) biological composite immobilized enzyme as an immobilizing agent to cure by means of microencapsulation the porous metal arsenate compound prepared by the transformation, which further reduces the leaching toxicity of the compound.

### DETAILED DESCRIPTION OF THE EMBODIMENT(S) OF THE DISCLOSURE

In order to make the object, technical solutions, and advantages of the embodiments of the present disclosure clearer, the technical solutions in the embodiments of the present disclosure will be described clearly and completely below. Obviously, the described embodiments are part, rather than all, of embodiments of the present disclosure. Based on the embodiments of the present disclosure, any other embodiments obtained by those of ordinary skill in the art without creative effort shall fall within the protection scope of the present disclosure.

Embodiment 1: the present embodiment provides a method for transforming an arsenic sulfide slag and curing and stabilizing the resulting compound by means of microencapsulation, including the following steps:

First step: preparing arsenic trioxide from the arsenic sulfide slag. First, the arsenic sulfide slag is added into a 50% concentration (mass fraction) sulfuric acid solution with a liquid-to-solid ratio by mass of 5:1, and stirred in a slurry tank for slurrying, with a stirring speed of 300rpm/min and a stirring time of 1 hour. After the slurrying, a slurry is pumped into a high-pressure reaction vessel, and a 70% concentration (mass fraction) sulfuric acid solution is added to adjust a liquid-solid ratio by mass in the reaction vessel into 7:1, and a temperature in the reaction vessel is 150° C. Oxygen is introduced into the reaction vessel, with an oxygen partial pressure controlled to be 0.6MPa, and the arsenic sulfide slag is oxdative pressure leached, with leaching reaction time of 3 hours. After the leaching reaction is completed, filtration is performed to achieve solid-liquid separation, and filtrate is pumped into a closed reaction vessel, and then reduced with sulfur dioxide as introduced, and cooled and crystallized, filtered and purified to obtain arsenic trioxide.

Second step: preparing 4-hydroxy-3-nitrophenylarsonic acid from the arsenic trioxide.

\hat{1} A little excess of aniline is added into a microwave heating reaction vessel and heated to 60° C. Later, arsenic acid solution produced by reaction of the arsenic trioxide with 0.5 mol/L excess hydrogen peroxide is uniformly added to the reaction vessel, and the heating is continued up to 165° C. for high-temperature synthesis of arsanilic acid.

\hat{2} Purification of the arsanilic acid: firstly, adding a 1 mol/L sodium hydroxide solution for alkaline layering, with a liquid-to-solid ratio (mass ratio) of 2:1; removing waste aniline remaining after the high-temperature synthesis reaction in removed floating liquid, and then adding l mol/L hydrochloric acid to neutralize the solution to a pH value of 3.5; adding water to achieve a volume ratio 1:2 of water to hydrochloric acid, and meanwhile heating the solution to boiling at 100° C. for hydrolysis to remove a by-product caused by the high-temperature synthesis of the arsanilic acid; after the hydrolysis is completed, moving the solution to a crystallization tank to be cooled and crystallized at 0° C.; after the arsanilic acid is fully crystallized, performing filtration and then crushing a filter cake and adding water again to produce a slurry, with a liquid-solid ratio 2:1 of water to arsanilic acid (mass ratio), and adding 0.1 mol/L sodium hydroxide solution to adjust a pH value to 6, and meanwhile heating the solution to 95° C., adding activated carbon for decolorization and impurity removal, and after completion of the impurity removal, cooling the solution to crystallize and freeze-dried at −10° C. to obtain arsanilic acid.

\hat{3} Synthesis of 4-hydroxy-3-nitrophenylarsonic acid: adding the arsanilic acid obtained above into a reaction vessel, first adding nitric acid, adjusting temperature, and then gradually adding sodium nitrite solution for diazotization at a temperature of 0° C., wherein a molar ratio of the arsanilic acid, the nitric acid and the sodium nitrite is 5:6:0.7; hydrolyzing and nitrating the solution after the diazotization is completed; holding the reaction at a constant temperature for 1 hour when the temperature rises to 55° C.; continuing to rise the temperature to 95° C. after nitrogen is surely released, and then stopping the temperature rising; and performing cooling and crystallization after completion of the hydrolysis and nitration, and precipitating a supernatant after the crystallization is completed, and obtaining 4-hydroxy-3-nitrophenylarsonic acid through suction filtration, freeze-drying, and pulverization.

Third step: synthesis of an iron-manganese dinuclear cluster metal arsenate compound having a porous structure. 10 mL of 0.1 mol/L hydrated iron perchlorate solution, 10 mL of 0.1 mol/L hydrated manganese perchlorate solution, and 10 mL of 0.1 mol/L ammonium citrate solution are mixed, and then a 20 mL hot water solution of 0.2 mol/L 4-hydroxy-3-nitrophenylarsonic acid, with a temperature of 80° C., is added, and a hydrochloric acid solution having a concentration of 3 M is added and stirred to adjust a pH value to 3.5, and then 2 g of F127 is added to adjust temperature of the solution to 60° C., and stirred for 24 hours for sol-gel reaction, and then evaporated in an oven at 80° C., and final dried gel is calcined at a temperature of 300° C. at a heating rate of 2° C./min for 4 hours to obtain an iron-manganese dinuclear cluster metal arsenate compound having a porous structure.

Fourth step: subjecting the iron-manganese dinuclear cluster metal arsenate compound having a porous structure to surface coating with silicon. The iron-manganese dinuclear cluster metal arsenate compound having a porous structure is uniformly dispersed into an alcohol aqueous solution with a volume ratio of 1:1, and stirred at a speed of 800 r/min in a 40° C. water bath, and a 3-aminopropyl triethoxy is added drop by drop, wherein mass ratio of the surface coating agent to the metal arsenate compound is 1:50, and the stirring speed is increased to 1000 r/min and kept for 1 hour. After completion thereof, suction filtration, washing, drying at a temperature of 80° C. and grinding are performed to obtain an iron-manganese dinuclear cluster metal arsenate compound having a porous structure that is surface coated with silicon.

Fifth step: preparing an Fe(0)/Al-SBA-15 mesoporous composite stabilizer.

\hat{1} First, preparation of Al-SBA-15 mesoporous material: weighing a certain amount of P123 and polyethylene glycol 4000 to dissolve in 100 mL of deionized water, stirring the same in a 40° C. water bath to achieve uniform dissolution, and then weighing a certain mass of aluminum nitrate and adding the same to the above solution to dissolve evenly and remain stable, and adding ethyl orthosilicate to the above solution, keeping stirring for 48 hours in a 40° C. water bath, and then transferring a reaction product to a reaction vessel for hydrothermal reaction for 24 hours at a temperature of 105° C., and finally performing suction and filtration, washing, drying and calcining at a temperature of 650° C. at a heating rate of 2° C./min for 6 hours to obtain Al-SBA-15 mesoporous material, wherein the P123, the polyethylene glycol 4000, the aluminum nitrate and the ethyl orthosilicate have a mass ratio of 40:20:1:40.

\hat{2} Preparation of an Fe(0)/Al-SBA-15 mesoporous composite stabilizer: fully dissolving a certain amount of ferrous sulfate into 100 mL of ethanol aqueous solution, then adding Al-SBA-15 mesoporous material powder in the above solution to be ultrasonically dispersed for 5 minutes, continuing to stir for 20 hours after completion thereof, and then transferring the same to a 500 mL three-necked flask, and adding nitrogen gas for 15 minutes to remove dissolved oxygen; then adding the polyethylene glycol 4000 to the above solution, stirring it for 30 minutes, and adjusting a pH value to 6 with 1 M NaOH solution; and finally, under continuous stirring, adding dropwise (1 drop/sec) 50 mL aqueous solution of potassium borohydride using a separatory funnel, and continuing the reaction for 40 minutes after the dropping, wherein a mass ratio of the ferrous sulfate, the Al-SBA-15 mesoporous material powder, the polyethylene glycol 4000, and the potassium borohydride is 4:4:1:2; after the reaction, when the mixture deposits to a bottom of a bottle, a precipitate is separated by centrifugation, and the precipitate obtained is washed alternately with deoxygenated deionized water and deoxygenated absolute ethanol for 3 times, dried and cooled in a vacuum drying oven at 70° C. to obtain the Fe(0)/Al-SBA-15 mesoporous composite stabilizer.

Sixth step: curing and stabilizing by means of microencapsulation. 55 parts by mass of iron-manganese dinuclear cluster metal arsenate compound having a porous structure to surface coating with silicon, 10 parts by mass of Fe(0)/Al-SBA-15 mesoporous composite stabilizer, 8 parts by mass of rectorite powder, and 3 parts by mass of TerraZyme bio-immobilized enzyme are weight for curing and stabilization, and a cured body is cured at 30° C. for 3 days. The cured stabilized product obtained by means of microencapsulation is tested in accordance with the identification standards for hazardous wastes identification for leaching toxicity (GB5085.3-2007), and the leaching toxicity test As is 0.08 ppm, which fully meets the landfill standards for safe landfills.

Embodiment 2: the present embodiment provides a method for transforming an arsenic sulfide slag and curing and stabilizing the resulting compound by means of microencapsulation, including the following steps:

First step: preparing arsenic trioxide from the arsenic sulfide slag. First, the arsenic sulfide slag is added into a 50% concentration (mass fraction) sulfuric acid solution with a liquid-to-solid ratio by mass of 5:1, and stirred in a slurry tank for slurrying, with a stirring speed of 500 rpm/min and a stirring time of 2 hours. After the slurrying, a slurry is pumped into a high-pressure reaction vessel, and a 70% concentration (mass fraction) sulfuric acid solution is added to adjust a liquid-solid ratio by mass in the reaction vessel into 7:1, and a temperature in the reaction vessel is 160° C. Oxygen is introduced into the reaction vessel, with an oxygen partial pressure controlled to be 0.7 MPa, and the arsenic sulfide slag is oxdative pressure leached, with leaching reaction time of 4 hours. After the leaching reaction is completed, filtration is performed to achieve solid-liquid separation, and filtrate is pumped into a closed reaction vessel, and then reduced with sulfur dioxide as introduced, and cooled and crystallized, filtered and purified to obtain arsenic trioxide.

Second step: preparing 4-hydroxy-3-nitrophenylarsonic acid from the arsenic trioxide.

\hat{1} A little excess of aniline is added into a microwave heating reaction vessel and heated to 70° C. Later, arsenic acid solution produced by reaction of the arsenic trioxide with 0.5 mol/L excess hydrogen peroxide is uniformly added to the reaction vessel, and the heating is continued up to 180° C. for high-temperature synthesis of arsanilic acid.

0 Purification of the arsanilic acid: firstly, adding a 1 mol/L sodium hydroxide solution for alkaline layering, with a liquid-to-solid ratio (mass ratio) of 2:1; removing waste aniline remaining after the high-temperature synthesis reaction in removed floating liquid, and then adding l mol/L hydrochloric acid to neutralize the solution to a pH value of 5.0; adding water to achieve a volume ratio 1:2 of water to hydrochloric acid, and meanwhile heating the solution to boiling at 105° C. for hydrolysis to remove a by-product caused by the high-temperature synthesis of the arsanilic acid; after the hydrolysis is completed, moving the solution to a crystallization tank to be cooled and crystallized at 10° C.; after the arsanilic acid is fully crystallized, performing filtration and then crushing a filter cake and adding water again to produce a slurry, with a liquid-solid ratio 2:1 of water to arsanilic acid (mass ratio), and adding 0.1 mol/L sodium hydroxide solution to adjust a pH value to 7, and meanwhile heating the solution to 95 , adding activated carbon for decolorization and impurity removal, and after completion of the impurity removal, cooling the solution to crystallize and freeze-dried at −10° C. to obtain arsanilic acid.

\hat{3} Synthesis of 4-hydroxy-3-nitrophenylarsonic acid: adding the arsanilic acid obtained above into a reaction vessel, first adding nitric acid, adjusting temperature, and then gradually adding sodium nitrite solution for diazotization at a temperature of 10° C., wherein a molar ratio of the arsanilic acid, the nitric acid and the sodium nitrite is 5:6:0.7; hydrolyzing and nitrating the solution after the diazotization is completed; holding the reaction at a constant temperature for 1 hour when the temperature rises to 75° C.; continuing to rise the temperature to 115° C. after nitrogen is surely released, and then stopping the temperature rising; and performing cooling and crystallization after completion of the hydrolysis and nitration, and precipitating a supernatant after the crystallization is completed, and obtaining 4-hydroxy-3-nitrophenylarsonic acid through suction filtration, freeze-drying, and pulverization.

Third step: synthesis of an iron-manganese dinuclear cluster metal arsenate compound having a porous structure. 10 mL of 0.1 mol/L hydrated iron perchlorate solution, 10 mL of 0.1 mol/L hydrated manganese perchlorate solution, and 10 mL of 0.1 mol/L ammonium citrate solution are mixed, and then a 20 mL hot water solution of 0.2 mol/L 4-hydroxy-3-nitrophenylarsonic acid, with a temperature of 80° C., is added, and a hydrochloric acid solution having a concentration of 3M is added and stirred to adjust a pH value to 5.5, and then 2 g of F127 is added to adjust temperature of the solution to 60° C., and stirred for 24 hours for sol-gel reaction, and then evaporated in an oven at 80° C., and final dried gel is calcined at a temperature of 400° C. at a heating rate of 2° C./min for 4 hours to obtain an iron-manganese dinuclear cluster metal arsenate compound having a porous structure.

Fourth step: subjecting the iron-manganese dinuclear cluster metal arsenate compound having a porous structure to surface coating with silicon. The iron-manganese dinuclear cluster metal arsenate compound having a porous structure is uniformly dispersed into an alcohol aqueous solution with a volume ratio of 1:1, and stirred at a speed of 800 r/min in a 40° C. water bath, and a 3-aminopropyl triethoxy is added drop by drop, wherein mass ratio of the surface coating agent to the metal arsenate compound is 1:50, and the stirring speed is increased to 1000 r/min and kept for 1 hour. After completion thereof, suction filtration, washing, drying at a temperature of 80° C. and grinding are performed to obtain an iron-manganese dinuclear cluster metal arsenate compound having a porous structure that is surface coated with silicon.

Fifth step: preparing an Fe(0)/Al-SBA-15 mesoporous composite stabilizer.

\hat{1} First, preparation of Al-SBA-15 mesoporous material: weighing a certain amount of P123 and polyethylene glycol 4000 to dissolve in 100 mL of deionized water, stirring the same in a 40° C. water bath to achieve uniform dissolution, and then weighing a certain mass of aluminum nitrate and adding the same to the above solution to dissolve evenly and remain stable, and adding ethyl orthosilicate to the above solution, keeping stirring for 48 hours in a 40° C. water bath, and then transferring a reaction product to a reaction vessel for hydrothermal reaction for 24 hours at a temperature of 115° C., and finally performing suction and filtration, washing, drying and calcining at a temperature of 650° C. at a heating rate of 2° C./min for 6 hours to obtain Al-SBA-15 mesoporous material, wherein the P123, the polyethylene glycol 4000, the aluminum nitrate and the ethyl orthosilicate have a mass ratio of 40:20:1:40.

\hat{2} Preparation of an Fe(0)/Al-SBA-15 mesoporous composite stabilizer: fully dissolving a certain amount of ferrous sulfate into 100 mL of ethanol aqueous solution, then adding Al-SBA-15 mesoporous material powder in the above solution to be ultrasonically dispersed for 5 minutes, continuing to stir for 20 hours after completion thereof, and then transferring the same to a 500 mL three-necked flask, and adding nitrogen gas for 15 minutes to remove dissolved oxygen; then adding the polyethylene glycol 4000 to the above solution, stirring it for 30 minutes, and adjusting a pH value to 6 with 1M NaOH solution; and finally, under continuous stirring, adding dropwise (1 drop/sec) 50 mL aqueous solution of potassium borohydride using a separatory funnel, and continuing the reaction for 40 minutes after the dropping, wherein a mass ratio of the ferrous sulfate, the Al-SBA-15 mesoporous material powder, the polyethylene glycol 4000, and the potassium borohydride is 4:4:1:2; after the reaction, when the mixture deposits to a bottom of a bottle, a precipitate is separated by centrifugation, and the precipitate obtained is washed alternately with deoxygenated deionized water and deoxygenated absolute ethanol for 3 times, dried and cooled in a vacuum drying oven at 70° C. to obtain the Fe(0)/Al-SBA-15 mesoporous composite stabilizer.

Sixth step: curing and stabilizing by means of microencapsulation. 65 parts by mass of iron-manganese dinuclear cluster metal arsenate compound having a porous structure to surface coating with silicon, 15 parts by mass of Fe(0)/Al-SBA-15 mesoporous composite stabilizer, 12 parts by mass of rectorite powder, and 5 parts by mass of TerraZyme bio-immobilized enzyme are weight for curing and stabilization, and a cured body is cured at 30° C. for 3 days. The cured stabilized product obtained by means of microencapsulation is tested in accordance with the identification standards for hazardous wastes identification for leaching toxicity (GB5085.3-2007), and the leaching toxicity test As is 0.02 ppm, which fully meets the landfill standards for safe landfills.

Embodiment 3: the present embodiment provides a method for transforming an arsenic sulfide slag and curing and stabilizing the resulting compound by means of microencapsulation, including the following steps:

First step: preparing arsenic trioxide from the arsenic sulfide slag. First, the arsenic sulfide slag is added into a 50% concentration (mass fraction) sulfuric acid solution with a liquid-to-solid ratio by mass of 5:1, and stirred in a slurry tank for slurrying, with a stirring speed of 450 rpm/min and a stirring time of 1.5 hours. After the slurrying, a slurry is pumped into a high-pressure reaction vessel, and a 70% concentration (mass fraction) sulfuric acid solution is added to adjust a liquid-solid ratio by mass in the reaction vessel into 7:1, and a temperature in the reaction vessel is 158° C. Oxygen is introduced into the reaction vessel, with an oxygen partial pressure controlled to be 0.67 MPa, and the arsenic sulfide slag is oxdative pressure leached, with leaching reaction time of 3.5 hours. After the leaching reaction is completed, filtration is performed to achieve solid-liquid separation, and filtrate is pumped into a closed reaction vessel, and then reduced with sulfur dioxide as introduced, and cooled and crystallized, filtered and purified to obtain arsenic trioxide.

Second step: preparing 4-hydroxy-3-nitrophenylarsonic acid from the arsenic trioxide.

\hat{1} A little excess of aniline is added into a microwave heating reaction vessel and heated. Later, arsenic acid solution produced by reaction of the arsenic trioxide with hydrogen peroxide is uniformly added to the reaction vessel, and the heating is continued up to 175° C. for high-temperature synthesis of arsanilic acid.

\hat{2} Purification of the arsanilic acid: firstly, adding a 1 mol/L sodium hydroxide solution for alkaline layering, with a liquid-to-solid ratio (mass ratio) of 2:1; removing waste aniline remaining after the high-temperature synthesis reaction in removed floating liquid, and then adding 1 mol/L hydrochloric acid to neutralize the solution to a pH value of 4.5; adding water to achieve a volume ratio 1:2 of water to hydrochloric acid, and meanwhile heating the solution to boiling at 103° C. for hydrolysis to remove a by-product caused by the high-temperature synthesis of the arsanilic acid; after the hydrolysis is completed, moving the solution to a crystallization tank to be cooled and crystallized at 7° C.; after the arsanilic acid is fully crystallized, performing filtration and then crushing a filter cake and adding water again to produce a slurry, with a liquid-solid ratio 2:1 of water to arsanilic acid (mass ratio), and adding 0.1 mol/L sodium hydroxide solution to adjust a pH value to 6.5, and meanwhile heating the solution to 95 , adding activated carbon for decolorization and impurity removal, and after completion of the impurity removal, cooling the solution to crystallize and freeze-dried at −10° C. to obtain arsanilic acid.

\hat{3} Synthesis of 4-hydroxy-3-nitrophenylarsonic acid: adding the arsanilic acid obtained above into a reaction vessel, first adding nitric acid, adjusting temperature, and then gradually adding sodium nitrite solution for diazotization at a temperature of 7° C., wherein a molar ratio of the arsanilic acid, the nitric acid and the sodium nitrite is 5:6:0.7; hydrolyzing and nitrating the solution after the diazotization is completed; holding the reaction at a constant temperature for 1 hour when the temperature rises to 65° C.; continuing to rise the temperature to 105° C. after nitrogen is surely released, and then stopping the temperature rising; and performing cooling and crystallization after completion of the hydrolysis and nitration, and precipitating a supernatant after the crystallization is completed, and obtaining 4-hydroxy-3-nitrophenylarsonic acid through suction filtration, freeze-drying, and pulverization.

Third step: synthesis of an iron-manganese dinuclear cluster metal arsenate compound having a porous structure. 10 mL of 0.1 mol/L hydrated iron perchlorate solution, 10 mL of 0.1 mol/L hydrated manganese perchlorate solution, and 10 mL of 0.1 mol/L ammonium citrate solution are mixed, and then a 20 mL hot water solution of 0.2 mol/L 4-hydroxy-3-nitrophenylarsonic acid, with a temperature of 80° C., is added, and a hydrochloric acid solution having a concentration of 3 M is added and stirred to adjust a pH value to 5.0, and then 2 gof F127 is added to adjust temperature of the solution to 60° C., and stirred for 24 hours for sol-gel reaction, and then evaporated in an oven at 80° C., and final dried gel is calcined at a temperature of 350° C. at a heating rate of 2° C./min for 4 hours to obtain an iron-manganese dinuclear cluster metal arsenate compound having a porous structure.

Fourth step: subjecting the iron-manganese dinuclear cluster metal arsenate compound having a porous structure to surface coating with silicon. The iron-manganese dinuclear cluster metal arsenate compound having a porous structure is uniformly dispersed into an alcohol aqueous solution with a volume ratio of 1:1, and stirred at a speed of 800 r/min in a 40° C. water bath, and a 3-aminopropyl triethoxy is added drop by drop, wherein mass ratio of the surface coating agent to the metal arsenate compound is 1:50, and the stirring speed is increased to 1000 r/min and kept for 1 hour. After completion thereof, suction filtration, washing, drying at a temperature of 80° C. and grinding are performed to obtain an iron-manganese dinuclear cluster metal arsenate compound having a porous structure that is surface coated with silicon.

Fifth step: preparing an Fe(0)/Al-SBA-15 mesoporous composite stabilizer.

\hat{1} First, preparation of Al-SBA-15 mesoporous material: weighing a certain amount of P123 and polyethylene glycol 4000 to dissolve in 100 mL of deionized water, stirring the same in a 40° C. water bath to achieve uniform dissolution, and then weighing a certain mass of aluminum nitrate and adding the same to the above solution to dissolve evenly and remain stable, and adding ethyl orthosilicate to the above solution, keeping stirring for 48 hours in a 40° C. water bath, and then transferring a reaction product to a reaction vessel for hydrothermal reaction for 24 hours at a certain temperature of 108° C., and finally performing suction and filtration, washing, drying and calcining at a temperature of 650° C. at a heating rate of 2° C./min for 6 hours to obtain Al-SBA-15 mesoporous material, wherein the P123, the polyethylene glycol 4000, the aluminum nitrate and the ethyl orthosilicate have a mass ratio of 40:20:1:40.

\hat{2} Preparation of an Fe(0)/Al-SBA-15 mesoporous composite stabilizer: fully dissolving a certain amount of ferrous sulfate into 100 mL of ethanol aqueous solution, then adding Al-SBA-15 mesoporous material powder in the above solution to be ultrasonically dispersed for 5 minutes, continuing to stir for 20 hours after completion thereof, and then transferring the same to a 500 mL three-necked flask, and adding nitrogen gas for 15 minutes to remove dissolved oxygen; then adding the polyethylene glycol 4000 to the above solution, stirring it for 30 minutes, and adjusting a pH value to 6 with 1 M NaOH solution; and finally, under continuous stirring, adding dropwise (1 drop/sec) 50 mL aqueous solution of potassium borohydride using a separatory funnel, and continuing the reaction for 40 minutes after the dropping, wherein a mass ratio of the ferrous sulfate, the Al-SBA-15 mesoporous material powder, the polyethylene glycol 4000, and the potassium borohydride is 4:4:1:2; after the reaction, when the mixture deposits to a bottom of a bottle, a precipitate is separated by centrifugation, and the precipitate obtained is washed alternately with deoxygenated deionized water and deoxygenated absolute ethanol for 3 times, dried and cooled in a vacuum drying oven at 70° C. to obtain the Fe(0)/Al-SBA-15 mesoporous composite stabilizer.

Sixth step: curing and stabilizing by means of microencapsulation. 60 parts by mass of iron-manganese dinuclear cluster metal arsenate compound having a porous structure to surface coating with silicon, 13 parts by mass of Fe(0)/Al-SBA-15 mesoporous composite stabilizer, 10 parts by mass of rectorite powder, and 4 parts by mass of TerraZyme bio-immobilized enzyme are weight for curing and stabilization, and a cured body is cured at 30° C. for 3 days. The cured stabilized product obtained by means of microencapsulation is tested in accordance with the identification standards for hazardous wastes identification for leaching toxicity (GB5085.3-2007), and the leaching toxicity test As is 0.04 ppm, which fully meets the landfill standards for safe landfills.

Embodiment 4: the present embodiment provides a method for transforming an arsenic sulfide slag and curing and stabilizing the resulting compound by means of microencapsulation, including the following steps:

First step: preparing arsenic trioxide from the arsenic sulfide slag. First, the arsenic sulfide slag is added into a 50% concentration (mass fraction) sulfuric acid solution with a liquid-to-solid ratio by mass of 5:1, and stirred in a slurry tank for slurrying, with a stirring speed of 350 rpm/min and a stirring time of 1.5 hours. After the slurrying, a slurry is pumped into a high-pressure reaction vessel, and a 70% concentration (mass fraction) sulfuric acid solution is added to adjust a liquid-solid ratio by mass in the reaction vessel into 7:1, and a temperature in the reaction vessel is 153° C. Oxygen is introduced into the reaction vessel, with an oxygen partial pressure controlled to be 0.65 MPa, and the arsenic sulfide slag is oxdative pressure leached, with leaching reaction time of 3.5 hours. After the leaching reaction is completed, filtration is performed to achieve solid-liquid separation, and filtrate is pumped into a closed reaction vessel, and then reduced with sulfur dioxide as introduced, and cooled and crystallized, filtered and purified to obtain arsenic trioxide.

Second step: preparing 4-hydroxy-3-nitrophenylarsonic acid from the arsenic trioxide.

\hat{1} A little excess of aniline is added into a microwave heating reaction vessel and heated. Later, arsenic acid solution produced by reaction of the arsenic trioxide with hydrogen peroxide is uniformly added to the reaction vessel, and the heating is continued up to 170° C. for high-temperature synthesis of arsanilic acid.

\hat{2} Purification of the arsanilic acid: firstly, adding a 1 mol/L sodium hydroxide solution for alkaline layering, with a liquid-to-solid ratio (mass ratio) of 2:1; removing waste aniline remaining after the high-temperature synthesis reaction in removed floating liquid, and then adding l mol/L hydrochloric acid to neutralize the solution to a pH value of 4.0; adding water to achieve a volume ratio 1:2 of water to hydrochloric acid, and meanwhile heating the solution to boiling at 102° C. for hydrolysis to remove a by-product caused by the high-temperature synthesis of the arsanilic acid; after the hydrolysis is completed, moving the solution to a crystallization tank to be cooled and crystallized at 3° C.; after the arsanilic acid is fully crystallized, performing filtration and then crushing a filter cake and adding water again to produce a slurry, with a liquid-solid ratio 2:1 of water to arsanilic acid (mass ratio), and adding 0.1 mol/L sodium hydroxide solution to adjust a pH value to 6.5, and meanwhile heating the solution to 95, adding activated carbon for decolorization and impurity removal, and after completion of the impurity removal, cooling the solution to crystallize and freeze-dried at −10° C. to obtain arsanilic acid.

\hat{3} Synthesis of 4-hydroxy-3-nitrophenylarsonic acid: adding the arsanilic acid obtained above into a reaction vessel, first adding nitric acid, adjusting temperature, and then gradually adding sodium nitrite solution for diazotization at a temperature of 3° C., wherein a molar ratio of the arsanilic acid, the nitric acid and the sodium nitrite is 5:6:0.7; hydrolyzing and nitrating the solution after the diazotization is completed; holding the reaction at a constant temperature for 1 hour when the temperature rises to 60° C.; continuing to rise the temperature to 100° C. after nitrogen is surely released, and then stopping the temperature rising; and performing cooling and crystallization after completion of the hydrolysis and nitration, and precipitating a supernatant after the crystallization is completed, and obtaining 4-hydroxy-3-nitrophenylarsonic acid through suction filtration, freeze-drying, and pulverization.

Third step: synthesis of an iron-manganese dinuclear cluster metal arsenate compound having a porous structure. 10 mL of 0.1 mol/L hydrated iron perchlorate solution, 10 mL of 0.1 mol/L hydrated manganese perchlorate solution, and 10 mL of 0.1 mol/L ammonium citrate solution are mixed, and then a 20 mL hot water solution of 0.2 mol/L 4-hydroxy-3-nitrophenylarsonic acid, with a temperature of 80° C., is added, and a hydrochloric acid solution having a concentration of 3 M is added and stirred to adjust a pH value to 4.0, and then 2 g of F127 is added to adjust temperature of the solution to 60° C., and stirred for 24 hours for sol-gel reaction, and then evaporated in an oven at 80° C., and final dried gel is calcined at a temperature of 340° C. at a heating rate of 2° C./min for 4 hours to obtain an iron-manganese dinuclear cluster metal arsenate compound having a porous structure.

Fourth step: subjecting the iron-manganese dinuclear cluster metal arsenate compound having a porous structure to surface coating with silicon. The iron-manganese dinuclear cluster metal arsenate compound having a porous structure is uniformly dispersed into an alcohol aqueous solution with a volume ratio of 1:1, and stirred at a speed of 800 r/min in a 40° C. water bath, and a 3-aminopropyl triethoxy is added drop by drop, wherein mass ratio of the surface coating agent to the metal arsenate compound is 1:50, and the stirring speed is increased to 1000 r/min and kept for 1 hour. After completion thereof, suction filtration, washing, drying at a temperature of 80° C. and grinding are performed to obtain an iron-manganese dinuclear cluster metal arsenate compound having a porous structure that is surface coated with silicon.

Fifth step: preparing an Fe(0)/Al-SBA-15 mesoporous composite stabilizer.

\hat{1} First, preparation of Al-SBA-15 mesoporous material: weighing a certain amount of P123 and polyethylene glycol 4000 to dissolve in 100 mL of deionized water, stirring the same in a 40° C. water bath to achieve uniform dissolution, and then weighing a certain mass of aluminum nitrate and adding the same to the above solution to dissolve evenly and remain stable, and adding ethyl orthosilicate to the above solution, keeping stirring for 48 hours in a 40° C. water bath, and then transferring a reaction product to a reaction vessel for hydrothermal reaction for 24 hours at a certain temperature of 110° C., and finally performing suction and filtration, washing, drying and calcining at a temperature of 650° C. at a heating rate of 2° C./min for 6 hours to obtain Al-SBA-15 mesoporous material, wherein the P123, the polyethylene glycol 4000, the aluminum nitrate and the ethyl orthosilicate have a mass ratio of 40:20:1:40.

\hat{2} Preparation of an Fe(0)/Al-SBA-15 mesoporous composite stabilizer: fully dissolving a certain amount of ferrous sulfate into 100 mL of ethanol aqueous solution, then adding Al-SBA-15 mesoporous material powder in the above solution to be ultrasonically dispersed for 5 minutes, continuing to stir for 20 hours after completion thereof, and then transferring the same to a 500 mL three-necked flask, and adding nitrogen gas for 15 minutes to remove dissolved oxygen; then adding the polyethylene glycol 4000 to the above solution, stirring it for 30 minutes, and adjusting a pH value to 6 with 1 M NaOH solution; and finally, under continuous stirring, adding dropwise (1 drop/sec) 50 mL aqueous solution of potassium borohydride using a separatory funnel, and continuing the reaction for 40 minutes after the dropping, wherein a mass ratio of the ferrous sulfate, the Al-SBA-15 mesoporous material powder, the polyethylene glycol 4000, and the potassium borohydride is 4:4:1:2; after the reaction, when the mixture deposits to a bottom of a bottle, a precipitate is separated by centrifugation, and the precipitate obtained is washed alternately with deoxygenated deionized water and deoxygenated absolute ethanol for 3 times, dried and cooled in a vacuum drying oven at 70° C. to obtain the Fe(0)/Al-SBA-15 mesoporous composite stabilizer.

Sixth step: curing and stabilizing by means of microencapsulation. 58 parts by mass of iron-manganese dinuclear cluster metal arsenate compound having a porous structure to surface coating with silicon, 12 parts by mass of Fe(0)/Al-SBA-15 mesoporous composite stabilizer, 9 parts by mass of rectorite powder, and 5 parts by mass of TerraZyme bio-immobilized enzyme are weight for curing and stabilization, and a cured body is cured at 30° C. for 3 days. The cured stabilized product obtained by means of microencapsulation is tested in accordance with the identification standards for hazardous wastes identification for leaching toxicity (GB5085.3-2007), and the leaching toxicity test As is 0.05 ppm, which fully meets the landfill standards for safe landfills.

The above are only preferred embodiments of the present disclosure, which are not used for limiting the present disclosure. For those skilled in the art, the present disclosure may be modified or varied in different ways. Any modification, equivalent replacement, improvement, etc., made within the spirit and principle of the present disclosure shall fall into the protection scope of the present disclosure.

What is claimed is:

1. A method for transforming an arsenic sulfide slag and curing and stabilizing the resulting compound by means of microencapsulation, comprising the following steps:
(1) preparing arsenic trioxide from the arsenic sulfide slag as a raw material;
(2) preparing 4-hydroxy-3-nitrophenylarsonic acid from the arsenic trioxide as a raw material;
(3) induce-preparing an iron-manganese dinuclear cluster metal arsenate compound having a porous structure by using the 4-hydroxy-3-nitrophenylarsonic acid through transformation and solvent evaporation;
(4) subjecting the iron-manganese dinuclear cluster metal arsenate compound having a porous structure to surface coating with silicon;
(5) synthesizing an Fe(0)/Al-SBA-15 mesoporous composite stabilizer by a hydrothermal reaction; and
(6) using the Fe(0)/Al-SBA-15 mesoporous composite stabilizer, an immobilized agent and an immobilizing enzyme for subjecting the iron-manganese dinuclear cluster metal arsenate compound coated with silicon to curing and stabilizing treatment by means of microencapsulation.

2. The method for transforming an arsenic sulfide slag and curing and stabilizing the resulting compound by means of microencapsulation according to claim 1, wherein the step (1) of preparing arsenic trioxide from the arsenic sulfide slag as a raw material comprises: firstly adding the arsenic sulfide slag into a sulfuric acid solution with a liquid-to-solid ratio by mass of 5:1, and stirring it in a slurry tank for slurrying, pumping a slurry after the slurrying into a high-pressure reaction vessel, and then adding a sulfuric acid solution to adjust a liquid-solid ratio into 7:1 by mass, and oxdative pressure leaching the arsenic sulfide slag, making filtration after the reaction, and pumping a filtrate into a closed reaction vessel, and then introducing sulfur dioxide to reduction, and performing cooling and crystallization, filtration and purification after the reduction to obtain arsenic trioxide.

3. The method for transforming an arsenic sulfide slag and curing and stabilizing the resulting compound by means of microencapsulation according to claim 1, wherein the step (2) of preparing 4-hydroxy-3-nitrophenylarsonic acid from the arsenic trioxide as a raw material comprises:
\hat{1} bringing the arsenic trioxide into reaction with hydrogen peroxide to produce an arsenic acid solution, and then reacting with an aniline to generate an arsanilic acid;
\hat{2} purifying the arsanilic acid obtained by the reaction; and
\hat{3} diazotizing, hydrolyzing and nitrating the purified arsanilic acid in sequence to prepare 4-hydroxy-3-nitrophenylarsonic acid.

4. The method for transforming an arsenic sulfide slag and curing and stabilizing the resulting compound by means of microencapsulation according to claim 1, wherein the step (3) of induce-preparing an iron-manganese dinuclear cluster metal arsenate compound having a porous structure by using the 4-hydroxy-3-nitrophenylarsonic acid through transformation and solvent evaporation comprises: mixing 10 mL hydrated iron perchlorate solution, 10 mL hydrated manganese perchlorate solution, and 10 mL complex solution, and then adding a water solution of 4-hydroxy-3-nitrophenylarsonic acid, and adding and stirring a 3M hydrochloric acid solution to adjust a pH value to 3.5-5.5, and then adding a template to adjust temperature of the solution to 60° C., and performing sol-gel reaction and then evaporation in an oven to obtain dry gel, and calcining at a temperature of 300-400° C., to obtain the iron-manganese dinuclear cluster metal arsenate compound having a porous structure.

5. The method for transforming an arsenic sulfide slag and curing and stabilizing the resulting compound by means of microencapsulation according to claim 1, wherein the step (4) of subjecting the iron-manganese dinuclear cluster metal arsenate compound having a porous structure to surface coating with silicon comprises: uniformly dispersing the iron-manganese dinuclear cluster metal arsenate compound having a porous structure into an alcohol aqueous solution with a volume ratio of 1:1, and adding a surface coating agent drop by drop in a state of being heated and stirred in a water bath, wherein a mass ratio of the surface coating agent to the metal arsenate compound is 1:50, and after completion of the coating, suction filtration, washing, drying and grinding are performed to obtain an iron-manganese dinuclear cluster metal arsenate compound having a porous structure that is surface coated with silicon.

6. The method for transforming an arsenic sulfide slag and curing and stabilizing the resulting compound by means of microencapsulation according to claim 1, wherein the step (5) of synthesizing an Fe(0)/Al-SBA-15 mesoporous composite stabilizer by a hydrothermal reaction comprises:
\hat{1} preparing Al-SBA-15 mesoporous material; and
\hat{2} preparing an Fe(0)/Al-SBA-15 mesoporous composite stabilizer.

7. The method for transforming an arsenic sulfide slag and curing and stabilizing the resulting compound by means of microencapsulation according to claim 6, wherein said preparing Al-SBA-15 mesoporous material comprises: weighing a certain amount of the template and surfactant to dissolve in 100 mL of deionized water, stirring the same in a water bath to achieve uniform dissolution, and then weighing a certain mass of aluminum nitrate and adding the same to the above solution to dissolve evenly, and adding ethyl orthosilicate to the above solution, keeping stirring for 48 hours in the water bath, and then transferring a reaction product to a reaction vessel for hydrothermal reaction for 24 hours, and finally performing suction and filtration, washing, drying and calcining at a temperature of 650° C. at a heating rate of 2° C./min for 6 hours to obtain Al-SBA-15 mesoporous material, wherein the template is P123, and the surfactant is polyethylene glycol 4000.

8. The method for transforming an arsenic sulfide slag and curing and stabilizing the resulting compound by means of microencapsulation according to claim 6, wherein said preparing an Fe(0)/Al-SBA-15 mesoporous composite stabilizer comprises: fully dissolving a certain amount of ferrous sulfate into 100 mL of ethanol aqueous solution, then adding Al-SBA-15 mesoporous material powder in the above solution to be ultrasonically dispersed, continuing to stir after completion thereof, and adding nitrogen gas to remove dissolved oxygen; then adding the polyethylene glycol 4000 and stirring, and adjusting a pH value to 6 with 1M NaOH solution; and finally, under continuous stirring, adding dropwise 50 mL aqueous solution of the reductant, and continuing the reaction for 40 minutes after the dropping, wherein a mass ratio of the ferrous sulfate, the Al-SBA-15 mesoporous material powder, the polyethylene glycol 4000, and the potassium borohydride is 4:4:1:2; and, after the reaction, separating a precipitate by centrifugation, and washing the obtained precipitate alternately with deoxygenated deionized water and deoxygenated absolute ethanol, drying and cooling the precipitate in a vacuum drying oven to obtain the Fe(0)/Al-SBA-15 mesoporous composite stabilizer.

9. The method for transforming an arsenic sulfide slag and curing and stabilizing the resulting compound by means of microencapsulation according to claim 1, wherein the step (6) of using the Fe(0)/Al-SBA-15 mesoporous composite stabilizer, an immobilized agent and an immobilizing enzyme for subjecting the iron-manganese dinuclear cluster metal arsenate compound coated with silicon to curing and stabilizing treatment by means of microencapsulation comprises: weighing 55-65 parts by mass of iron-manganese dinuclear cluster metal arsenate compound having a porous structure to surface coating with silicon, 10-15 parts by mass of Fe(0)/Al-SBA-15 mesoporous composite stabilizer, 8-12 parts by mass of immobilizing agent, and 3-5 parts by mass of immobilized enzyme are weight for curing and stabilization, and a cured body is cured at 30° C. for 3 days.

10. The method for transforming an arsenic sulfide slag and curing and stabilizing the resulting compound by means of microencapsulation according to claim 1, wherein the immobilizing agent used is rectorite powder with an average particle size of 5 μm; and the immobilized enzyme is TerraZyme bio-immobilized enzyme.
